(19) United States
(12) Patent Application Publication
(10) Pub. No.: US 2009/0011050 A1
(43) Pub. Date: Jan. 8, 2009

(54) Skin Treatment Composition And Method

(76) Inventor: Dennis Simmons, Shawnee Mission, KS (US)

Correspondence Address: LATHROP & GAGE LC, 2345 GRAND AVENUE, SUITE 2800, KANSAS CITY, MO, 64108, US

(21) Appl. No.: 12/166,147

(22) Filed: Jul. 1, 2008

Related U.S. Application Data
(60) Provisional application No. 60/948,345, filed on Jul. 6, 2007.

(51) Int. Cl. A61K 33/14

(52) U.S. Cl. 424/678

(57) ABSTRACT

Disclosed is a composition which is provided as a cream. One use for the product is for removing urushiol oil from the dermal layer of the skin using a combination of other oils. The composition may include all or some of Apricot oil (kernel oil), Avocado oil, Dead Sea Salts, Eucalyptus (essential oil), Grapefruit oil (pink, essential oil), Grapeseed oil (essential oil), Jojoba oil (organic), Lemon oil (essential oil), Orange oil juicy flavor oil), Peppermint (essential oil), Rosemary oil (essential oil), Spearmint oil (essential oil), Vitamin E oil (T-50 all natural); and Walnut oil.

### RELATED APPLICATIONS

This application claims the benefit of U.S. Provisional No. 60/948,345 filed Jul. 6, 2007, the disclosure of which is incorporated herein by reference.

### BACKGROUND OF THE INVENTION

1. Field of the Invention

The present invention relates to the field of skin care. More specifically, one embodiment of the present invention is directed to a method and composition for treating poison ivy and other similar allergic reactions.

2. Description of the Related Art

The oleoresin urushiol, which flows through the Poison Ivy plant and its relatives, causes an allergic reaction (a red, itchy rash). Urushiol is a sticky, clear oil which contains catechols and other phenolic resins that act as a powerful hapten (a substance that does not stimulate antibody formation but reacts selectively in vitro with an antibody). Urushiol resin remains stable, even in dead or dried plants, and therefore is equally hazardous in the winter as in the summer.

Estimates state that 50-70% of the U.S.A. population is allergic to urushiol, and would acquire the rash on casual contact. Prolonged exposure may induce an allergy. The reaction ranges from mild to severe, and sensitivity can develop later in life. If a subject is allergic to Poison Ivy, they are also allergic to poison oak and poison sumac, and may be allergic to cashew nut shell oil, mango fruit peels, and Japanese lacquer. A person is not allergic to Poison Ivy the first time they touch it, but can become allergic as the skin sensitizes to the resin.

Conventionally, some have attempted to use soap to remove the troublesome oil. But soap doesn't work because Urushiol oil penetrates the top layer of skin and binds to the dermal layer. Limited success can be achieved in removing urushiol oil from the skin prior to bonding. But bonding takes place in as little as 3 minutes. And the average time required is 30 minutes.

Soaps are sodium or potassium fatty acids salts, produced from the hydrolysis of fats in a chemical reaction called saponification. Each soap molecule has a long hydrocarbon chain, sometimes called its ‘tail’, with a carboxylate ‘head’. In water, the sodium or potassium ions float free, leaving a negatively-charged head.

Soap is an excellent cleanser because of its ability to act as an emulsifying agent. An emulsifier is capable of dispersing one liquid into another immiscible liquid. This means that while oil (which attracts dirt) doesn't naturally mix with water, soap can suspend oil/dirt in such a way that it can be removed. The organic part of a natural soap is a negatively-charged, polar molecule. Its hydrophilic (water-loving) carboxylate group (—CO2) interacts with water molecules via ion-dipole interactions and hydrogen bonding. The hydrophobic (water-fearing) part of a soap molecule, its long, nonpolar hydrocarbon chain, does not interact with water molecules. The hydrocarbon chains are attracted to each other by dispersion forces and cluster together, forming structures called micelles. In these micelles, the carboxylate groups form a negatively-charged spherical surface, with the hydrocarbon chains inside the sphere. Because they are negatively charged, soap micelles repel each other and remain dispersed in water. But even though soaps are normally effective in the removing oils, once urushiol oil becomes bonded into the subdermal regions, the soaps will not be able to detach it.

Solvents are also ineffective. Because urushiol oil penetrates the top layer of skin and binds to the dermal layer after only a few minutes, limited success can be achieved by using solvents before bonding occurs. Removing the urushiol with solvents is very much like removing the remaining paint from a paint brush. The urushiol can be rinsed off with a solvent prior to bonding. But after bonding, using a solvent proves worthless much like trying to remove cured paint on a surface with paint thinner. Similarly, once urushiol oil has bonded, solvents will be ineffective.

Perhaps the most common solvent in everyday life is water. Many other solvents are organic compounds, such as benzene, tetrachloroethylene or turpentine. Solvents can be broadly classified into polar and non-polar solvents. Common polar solvents include water and ethanol. Generally polar or ionic compounds will only dissolve in polar solvents. In chemistry, a common rule for determining if a solvent will dissolve a given solute is “like dissolves like.” Solvents composed of polar molecules, such as water, dissolve other polar molecules, such as table salt, while nonpolar solvents, such as gasoline, dissolve nonpolar substances such as wax. The degree that a solvent dissolves a given solute is known as its solubility. Ethyl alcohol is highly soluble in water, for example. Vinegar is very insoluble in oil, and the two substances will quickly separate into two layers even after being shaken well. But these solvents, although used in some instances to treat poison ivy, are in reality largely ineffective in removing urushiol oil that has become subdermally attached.

Therefore, there is a need in the art for removing urushiol oil in consideration of the limitations discussed above.

### DETAILED DESCRIPTION

The present invention is a composition which is provided as a creamy composition. The composition solves the need in the art by immediately and permanently removing the urushiol oil from the dermal layer of the skin of the user. In one embodiment, the product includes all or some of: Apricot oil (kernel oil), Avocado oil, Dead Sea Salts, Eucalyptus (essential oil), Grapefruit oil (pink, essential oil), Grapeseed oil (essential oil), Jojoba oil (organic), Lemon oil (essential oil), Orange oil (juicy flavor oil), Peppermint (essential oil), Rosemary oil (essential oil), Spearmint oil (essential oil), Vitamin E oil (T-50 all natural); and Walnut oil. One embodiment for the composition in particular includes the following ingredients as a percentage by volume:

| | | Range of |
|---|---|---|
| | Percentage in | inclusion in |
| | One | Some |
| Ingredient List | Embodiment | Embodiments |
| Apricot oil (kernel oil) | 3.37% | 1-4% |
| Avocado oil | 3.00% | 1-3% |
| Dead Sea Salts | 72.31% | 70-75% |
| Eucalyptus (essential oil) | 2.00% | .5-2 |
| Grapefruit oil (pink, essential oil) | 0.13% | .1-.13 |
| Grapeseed oil (essential oil) | 5.00% | 1-4% |
| Jojoba oil (organic) | 1.50% | 1-1.5% |
| Lemon oil (essential oil) | 1.00% | .4-1 |
| Orange oil (juicy flavor oil) | 0.75% | .25-.75 |
| Peppermint (essential oil) | 4.00% | 1-4% |
| Rosemary oil (essential oil) | 0.50% | .1-.5 |
| Spearmint oil (essential oil) | 0.25% | .1-.25 |
| Vitamin E oil (T-50 all natural) | 0.19% | .1-.19 |
| Walnut oil | 6.00% | 2-6% |
| Total | 100.00% | |

These ingredients are mixed together using a mixing hopper and then included in a container for distribution to consumers. All the ingredients are commercially available. Although the oils are shown having particular percentages and then ranges, it should be understood that each could be either eliminated, substituted by another like oil, or included in the range of 0-100% and still fall within the objectives of the disclosed embodiments. Similarly, the Dead Sea Salts could be omitted, substituted by another salt, or be included in any percentage. The exact composition of these salts is somewhat a mystery, but it has been estimated that they comprise approximately, in terms of w/w % composition of the salt, as anhydrous chlorides, calcium chloride (CaCl2) 14.4%, potassium chloride (KCl) 4.4%, magnesium chloride (MgCl2) 50.8% and sodium chloride (common salt, NaCl) 30.4%.

Once received by a consumer/user, the tube should be shaken or otherwise agitated vigorously. In the preferred embodiment, the tube should be shaken vigorously for about 15 seconds.

Next, the thoroughly shaken composition is removed from the container, and then a very small amount of it deposed onto a finger or damp cloth. Then the user rubs the coated finger or cloth on the inflamed area for about but not more than 15 seconds.

When it is rubbed on the inflamed area, the applied oil mixture actually breaks away the urushiol molecules out of the cavities of the dermal layer of the skin. When the atoms of the selected oils rub against one another, they create wavelengths of energy in response to the rubbing at very precisely timed intervals that essentially build upon themselves to create powerful enough forces to break the urushiol apart. The process relies on electrical, thermal, and acoustical processes.

As the different atoms of oils are rolling over each other, they create micro push-pulls (or waves) that in essence vibrate the skin at an extremely minute level. The crystals of the Dead Sea Salts scratch at the urushiol resin allowing for other oils that have similar electrical properties to attach to the urushiol resin.

As the oils continue to rub against each other with the crystals of the Dead Sea salts, they generate heat. As the surrounding area heats up, the skin stretches at a different rate then the urushiol resin, creating more gaps for the oils to penetrate and further surround the urushiol resin, dislodging it by sucking it out; a result of the micro push-pulls on the area.

The final step is to rinse the area with water, no soap, leaving the oils to stay attached only to the urushiol resin. If soap is used, the oils can free themselves from the urushiol resin, and attach themselves to the soap since its attraction is greater to the soap then the urushiol resin. Since oil and water don't mix, as the water is rinsed over the area, the oils ball up (congregate around each other) and flush themselves off as if they were on a slipper slide.

In addition to removing the urushiol resin, after the bond between the urushiol resin and subdermal layer of the skin to which it is attached is broken apart, other oils are left in the newly created cavities of the dermal layer of the skin (where the urushiol resin resided) tightly forming a vacuumed, lubricated bond. An additional benefit is that this attachment has shown to smooth crow's feet, light lines and wrinkles.

The lubricated bond created minimizes scarring because (i) moisture can't penetrate and dry out the inflamed area, and (ii) the oils provide for a smoother surface for the skin to grow back. In addition, the user is less likely to itch or scratch the inflamed area because the oils provide an insulating effect for the nerve endings (the itch) while the skin heals.

The composition of the present invention will also leave a protective film layer that will protect your skin from future allergic reactions from urushiol, and has shown to be effective in reducing symptoms of other skin conditions such as acne, eczema, seborrhea and psoriasis.

1. A composition for treating a skin condition on an animal, said compound consisting essentially of the following ingredients as a percentage by volume:

| | |
|---|---|
| Apricot oil (kernel oil) | 1-4% |
| Avocado oil | 1-3% |
| Dead Sea Salts | 70-75% |
| Eucalyptus (essential oil) | .5-2% |
| Grapefruit oil (pink, essential oil) | .1-.13% |
| Grapeseed oil (essential oil) | 1-4% |
| Jojoba oil (organic) | 1-1.5% |
| Lemon oil (essential oil) | .4-1% |
| Orange oil (juicy flavor oil) | .25-.75% |
| Peppermint (essential oil) | 1-4% |
| Rosemary oil (essential oil) | .1-.5% |
| Spearmint oil (essential oil) | .1-.25% |
| Vitamin E oil (T-50 all natural) | .1-.19%; and |
| Walnut oil | 2-6%. |

2. The composition of claim 1 wherein said composition is adapted for administration on a human.

3. The composition of claim 1 wherein said skin condition is urushiol oil contamination.

4. The composition of claim 1 consisting essentially of the following ingredients at about the percentages by volume shown below:

| | |
|---|---|
| Apricot oil (kernel oil) | 3.37% |
| Avocado oil | 3.00% |
| Dead Sea Salts | 72.31% |
| Eucalyptus (essential oil) | 2.00% |
| Grapefruit oil (pink, essential oil) | 0.13% |
| Grapeseed oil (essential oil) | 5.00% |
| Jojoba oil (organic) | 1.50% |
| Lemon oil (essential oil) | 1.00% |
| Orange oil (juicy flavor oil) | 0.75% |
| Peppermint (essential oil) | 4.00% |
| Rosemary oil (essential oil) | 0.50% |
| Spearmint oil (essential oil) | 0.25% |
| Vitamin E oil (T-50 all natural) | 0.19%; and |
| Walnut oil | 6.00%. |

5. A method for treating a skin condition, said method comprising:
mixing a plurality of oils along with a salt together to form a compound in a container;
adapting said compound for topical applications;
instructing a user to rubs the compound over an afflicted area for a period of time until heat is generated; and
further instructing said user to rinse the area after said compound has been rubbed over said afflicted area for said period of time.

6. The method of claim 5 wherein said skin condition is one of aging, acne, eczema, seborrhea and psoriasis.

7. The method of claim 6 in which the skin condition is urushiol oil contamination.

8. A composition for use as a creamy composition for removing urushiol oil from a dermal layer of skin of a user, said composition further comprising a plurality of oils and at least one salt, said oils selected from the list comprising: Apricot oil (kernel oil), Avocado oil, Eucalyptus (essential oil), Grapefruit oil (pink, essential oil), Grapeseed oil (essential oil), Jojoba oil (organic), Lemon oil (essential oil), Orange oil (juicy flavor oil), Peppermint (essential oil), Rosemary oil (essential oil), Spearmint oil (essential oil), Vitamin E oil (T-50 all natural); and Walnut oil.
